(19) United States
(12) Patent Application Publication
(10) Pub. No.: US 2024/0009071 A1
(43) Pub. Date: Jan. 11, 2024

(54) THERMOTHERAPY DEVICE

(71) Applicant: CERAGEM CO., LTD., Cheonan-si, Chungcheongnam-do (KR)

(72) Inventors: Dong Myoung Lee, Asan-si, Chungcheongnam-do (KR); Keun Young Paek, Cheonan-si, Chungcheongnam-do (KR); Sang Ho Choi, Cheonan-si, Chungcheongnam-do (KR)

(21) Appl. No.: 18/254,505

(22) PCT Filed: Nov. 24, 2021
(86) PCT No.: PCT/KR2021/017411

§ 371 Date: May 25, 2023

(30) Foreign Application Priority Data
Nov. 26, 2020 (KR) 10-2020-0160967

(51) Int. Cl. A61H 15/02; A61H 15/00

(52) U.S. Cl. CPC A61H 15/02; A61H 15/0078; A61H 2201/0146; A61H 2201/0207; A61H 2201/0235; A61H 2203/0456

(57) ABSTRACT

The present invention relates to a thermotherapy device. More specifically, the present invention relates to a thermotherapy device which can receive a current from a power supply part even when a heating part for heating a ceramic part rotates together with the ceramic part. To this end, the thermotherapy device comprises: the ceramic part having an inner space formed therein; the heating part inserted in the inner space and having a heating element, generating heat so as to heat the ceramic part, and a transfer member transferring the heat generated by the heating element to the ceramic part; the power supply part supplying a current to the heating part; and a support part supporting the ceramic part, wherein the heating part can rotate relative to the power supply part such that the heating part rotates together with the ceramic part.

### TECHNICAL FIELD

The present invention relates to a thermotherapy device. More specifically, it relates to a thermotherapy device which is capable of receiving a current from a power supply part even in a state where the heating part for heating a ceramic part rotates together with the ceramic part.

### BACKGROUND ART

Conventionally, thermotherapy devices that relieve acute or chronic pain occurring in the muscles and nervous tissue of the spine caused by continuing work for a long period of time in an inappropriate posture or habituating this posture for a long period of time, move along the body part to improve the blood circulation of the body or relieve momentary muscle stiffness, and improve blood circulation through stimulation by heating in the area where pain occurs have been widely used.

The conventional thermotherapy device used for such thermotherapy performs massage while a heating ceramic is moved in the longitudinal direction along the user's body, and the heating ceramic is configured to rotate in the process of repeatedly reciprocating the entire moving section to massage the user's body. This is to configure a heating ceramic to rotate naturally due to the friction of a cover, because if the heating ceramic does not rotate, the friction between the heating ceramic and the cover may be maximized, and the cover may be quickly worn out.

In the conventional case, a heating element in a non-rotating state which is connected to a power source is inserted into the heating ceramic in order to heat the rotating heating ceramic, and the heating ceramic is configured to be spaced apart from the heating element such that the rotating heating ceramic can rotate relative to the heating element in a non-rotating state.

However, as the heating ceramic and the heating element are disposed to be spaced apart from each other, the heat generated from the heating element is not y transferred, and thus, there is a problem in that the thermotherapy effect is reduced.

Therefore, there is a need for improvement in these areas.

(Patent Document 1) Korean Registered Patent No. 2002-0039608 (published on May 27, 2002)

### DISCLOSURE

### Technical Tasks

The technical problem to be solved in the present invention is directed to solving the problems of the related art described above, and it is directed to providing a thermotherapy device which is capable of receiving a current from a power supply part even in a state where the heating part for heating a ceramic part rotates together with the ceramic part.

The technical problems to be solved in the present invention are not limited thereto, and other technical problems that are not mentioned will be clearly understood by those skilled in the art from the following description.

### Technical Solution

In order to solve the above-described technical problems, the thermotherapy device according to the present invention includes a ceramic part having an inner space formed therein; a heating part which is inserted in the inner space and has a heating element for generating heat so as to heat the ceramic part, and a transfer member for transferring the heat generated by the heating element to the ceramic part; a power supply part for supplying a current to the heating part; and a support part for supporting the ceramic part, wherein the heating part can rotate relative to the power supply part such that the heating part rotates together with the ceramic part.

In this case, the transfer member may include a first transfer body and a second transfer body that are disposed to face each other, and the heating element may be provided between the first transfer body and the second transfer body.

In this case, the power supply part may be provided with a first electrode which is disposed on one side of the ceramic part and a second electrode which is disposed on the other side of the ceramic part.

In this case, an insulating member may be provided between the first transfer body and the second transfer body.

In this case, the insulating member may be formed with an insertion groove into which the heating element is inserted and disposed, and both side surfaces of the heating element may be exposed to contact with the first transfer body and the second transfer body.

In this case, a first conductive surface which electrically contacts the first electrode may be formed on the first transfer body, and a second conductive surface which electrically contacts the second electrode may be formed on the second transfer body.

In this case, the first conductive surface may be formed to extend to surround the second transfer body, wherein the second conductive surface may be formed to extend to surround the first transfer body, and wherein the insulating member may be formed with a base surface which is disposed between the first transfer body and the second transfer body, and bent surfaces which are disposed between the first conductive surface and the second transfer body and between the second conductive surface and the first transfer body, respectively.

In this case, a first electrode plate which electrically contacts the first electrode may be formed on the first transfer body, and a second electrode plate which electrically contacts the second electrode may be formed on the second transfer body.

In this case, a first head which is in contact with the first electrode and a first body which is in contact with one side surface of the heating element may be formed on the first electrode plate, and a second head which is in contact with the second electrode and a second body which is in contact with the other side surface of the heating element may be formed on the second electrode plate.

In this case, a first support surface which extends to surround the second transfer body and has a first through-hole formed such that the first head is exposed to the outside may be formed on the first transfer body, and a second support surface which extends to surround the first transfer body and has a second through-hole formed such that the second head is exposed to the outside may be formed on the second transfer body.

In this case, the insulating member may be formed with a base surface which is disposed between the first transfer body and the second transfer body, and bent surfaces which are disposed between the first support surface and the second transfer body and between the second support surface and the first transfer body, respectively.

In this case, the heating element may be formed to protrude by a certain height such that both side surfaces are exposed.

In this case, a first protrusion may be formed on the first transfer body to press one side surface of the heating element, and a second protrusion may be formed on the second transfer body to press the other side surface of the heating element.

In this case, the heating part may be provided with an elastically deformable member that presses the inner peripheral surface of the ceramic part.

### Advantageous Effects

Since the thermotherapy device of the present invention having the above configuration is configured such that the heating part for heating the ceramic part rotates together with the ceramic part, the heat generated in the heating part is smoothly transferred to the ceramic part, as the ceramic part and the heating part are disposed to contact with each other, thereby improving the thermotherapy effect.

In addition, as heat is smoothly transferred from the heating part to the ceramic part, heat loss is minimized, and thus, the power consumption efficiency of the thermotherapy device is improved.

Moreover, since the current is stably supplied even in a state where the heating part rotating together with the ceramic part rotates relative to the power supply part, it is possible to secure the operation stability of the thermotherapy device.

It should be understood that the effects of the present invention are not limited to the above-described effects, and include all effects that can be inferred from the configuration of the invention described in the detailed description or claims of the present invention.

### DESCRIPTION OF DRAWINGS

FIG. 1 is a cross-sectional view showing a heat treatment device according to an exemplary embodiment of the present invention.

FIG. 2 is a cross-sectional view illustrating a state in which a ceramic part and a heating part are coupled according to an exemplary embodiment of the present invention.

FIG. 3 is a cross-sectional view illustrating a state in which the ceramic part and the heating part are disassembled according to an exemplary embodiment of the present invention.

FIG. 4 is a perspective view illustrating a heating part according to an exemplary embodiment of the present invention.

FIG. 5 is an exploded perspective view illustrating a heating part according to an exemplary embodiment of the present invention.

FIG. 6 is a cross-sectional view illustrating a conductive surface which is formed on a heating part according to an exemplary embodiment of the present invention.

FIG. 7 is a perspective view illustrating a heating part according to another exemplary embodiment of the present invention.

FIG. 8 is an exploded perspective view illustrating a heating part according to another exemplary embodiment of the present invention.

FIG. 9 is a cross-sectional view illustrating the coupling state of a heating element and a transfer member according to still another exemplary embodiment of the present invention.

FIG. 10 is a cross-sectional view illustrating a state in which the ceramic part and the heating part are coupled according to still another exemplary embodiment of the present invention.

FIG. 11 is a side view illustrating a heating part according to still another exemplary embodiment of the present invention.

FIG. 12 is a flowchart illustrating the process of coupling a heating part to a ceramic part according to an exemplary embodiment of the present invention.

### MODES OF THE INVENTION

Hereinafter, with reference to the accompanying drawings, the exemplary embodiments of the present invention will be described in detail so that those of ordinary skill in the art can easily practice the present invention. The present invention may be embodied in many different forms and is not limited to the exemplary embodiments described herein. In order to clearly describe the present invention in the drawings, parts that are irrelevant to the description are omitted, and the same reference numerals are assigned to the same or similar components throughout the specification.

In the present specification, terms such as “include” or “have” are intended to designate that a feature, number, step, operation, component, part or combination thereof described in the specification exists, but it should be understood that it does not preclude the possibility of the presence or addition of one or more numbers, steps, operations, components, parts or combinations thereof. In addition, when a part of a layer, film, region, plate and the like is said to be “on” another part, this includes not only cases where the other part is “directly on”, but also cases where there is another part therebetween. Conversely, when a part of a layer, film, region, plate and the like is said to be “under” another part, this includes not only cases where it is “directly under” another part, but also cases where there is another part therebetween.

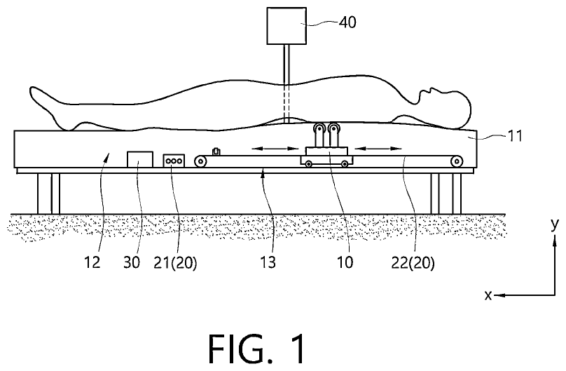

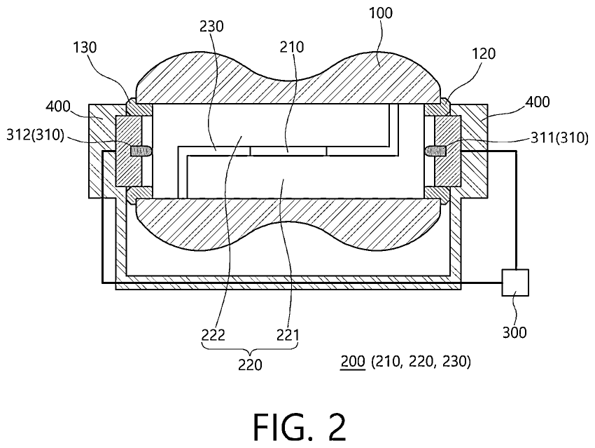

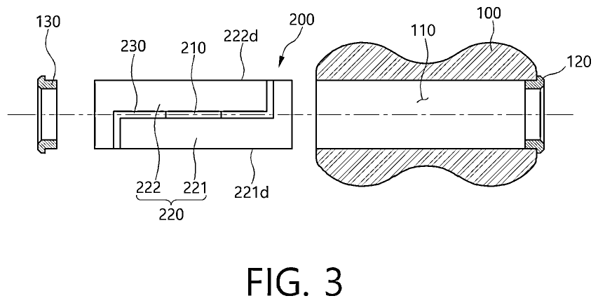

FIG. 1 is a cross-sectional view showing a heat treatment device according to an exemplary embodiment of the present invention, FIG. 2 is a cross-sectional view illustrating a state in which a ceramic part and a heating part are coupled according to an exemplary embodiment of the present invention, and FIG. 3 is a cross-sectional view illustrating a state in which the ceramic part and the heating part are disassembled according to an exemplary embodiment of the present invention.

As illustrated in FIG. 1, the thermotherapy device according to an exemplary embodiment of the present invention includes a ceramic module 10, a driving part 20 for moving the ceramic module 10, a control part 30 for controlling the operation of the driving part 20, and an input part 40 for inputting a desired thermotherapy pattern by the user.

In this case, such a thermotherapy device may include a main mat 11 which is used for the user's upper body and the spine portion thereof, and an auxiliary mat 12 which is used for the user's lower body part as a target. In addition, it may include a mounting part 13 for placing and supporting the main mat 11 and the auxiliary mat 12 as necessary.

The ceramic module 10 may massage the spine while moving in the longitudinal direction (x) along the user's spine, and the ceramic module 10 may provide a thermal compress and massage effect to the user by using high-temperature heat generated by using a current supplied from a power supply part 300 to be described below.

In this case, the ceramic module 10 may be configured to provide the user with a thermal compress and massage effect by using not only high-temperature heat but also far-infrared rays.

The ceramic part 100 provided in the ceramic module 10 may be formed in a roller type, but the present invention is not limited thereto, and if the ceramic part 100 is configured to rotate while the ceramic module 10 is moved, various shapes and structures are possible. In addition, when the ceramic part 100 is formed of a material such as ceramic, far-infrared rays are generated in the process of using the thermotherapy device, and thus, the thermotherapy effect may be improved. However, the present invention is not necessarily limited to these materials, and it may be formed of other materials as long as they can transfer heat to the user's body and provide a thermotherapy effect.

As illustrated in FIG. 2, the ceramic module 10 includes a ceramic part 100 having an inner space 110 formed therein, a heating part 200 which is inserted into the inner space 110 and has a heating element 210 for generating heat such that the ceramic part 100 is heated and a transfer member 220 for transferring the heat generated from the heating element 210 to the ceramic part 100, a power supply part 300 for supplying a current to the heating part 200, and a support part 400 for supporting the ceramic part 100.

Herein, a PTC heater may be used as the heating element 210, but the present invention is not limited thereto, and a lamp which is capable of heating by supplying a current or various heating elements may be used.

In addition, the driving part 20 may include a first driving member for moving the ceramic module 10 in the longitudinal direction (x) of the user. The first driving member may include a driving motor 21 and a transport member 22 for reciprocating the ceramic module 10.

The driving motor 21 receives a current to rotate, and the transport member 22 is connected to the driving motor 21 and transmits the rotational force according to the rotation of the driving motor 21 to move the ceramic module 10.

The transport member 22 is connected to the ceramic module 10, and according to the forward or reverse rotation of the driving motor 21, it is used to transfer the ceramic module 10 in one side direction or the other side direction along the longitudinal direction (x) of the user.

The transport member 22 may be selectively used among a transfer belt, a transfer chain and a transfer rope, but the present invention is not limited thereto, and various means for transporting an object by using the driving force of the driving motor 21 may be used, such as a lead screw or a method of using a rack and pinion.

The driving motor 21 may be configured to provide a driving force while being spaced apart from the magnetic module 10 or to provide a driving force while being inserted into the magnetic module 10.

Moreover, the driving part 20 may include a second driving member for raising the vertical height of the ceramic module 10 such that the pressing force is applied to the user or lowering the vertical height of the ceramic module 10 such that the pressing force is removed.

In this case, as illustrated in FIG. 2, in the thermotherapy process, the heating part 200 is configured to rotate together so as to rotate with the ceramic part 100, and thus, as the ceramic part 100 and the heating part 200 are disposed to be in contact with each other, the heat generated from the heating part 200 is smoothly transferred to the ceramic part 100, thereby improving the thermotherapy effect.

The heating part 200 may be configured to evenly contact the entire inner peripheral surface of the inner space 110 formed in the ceramic part 100, but the present invention is not necessarily limited thereto, and if the heat generated from the heating part 200 can be smoothly transferred to the ceramic part 100, it is also possible to configure the same to contact each other only at certain parts.

In this way, as heat is smoothly transferred from the heating part 200 to the ceramic part 100, heat loss is minimized and the power consumption efficiency of the thermotherapy device is improved, and the heating part 200 is stably supplied with a current through the power supply part 300 while rotating relative to the power supply part 300 such the thermotherapy device can operate stably. In this case, although relative rotation of the heating part 200 and the power supply part 300 may occur as the power supply part 300 is fixed in a non-rotating state, depending on some cases, the power supply part 300 also rotates at a constant speed, but relative rotation may occur between the heating part 200 and the power supply part 300 as it rotates slower than the rotational speed of the heating part 200 (low-speed rotation state).

The power supply part 300 may be provided with an electrode member 310 to be described below, and the electrode member 310 may be configured to smoothly supply a current even in a state where the heating part 200 rotates together with the ceramic part 100.

As illustrated in FIG. 3, a first bushing 120 is provided on one side of the ceramic part 100, and a second bushing 130 is provided on the other side of the ceramic part 100 such that the ceramic part 100 is rotatably supported by the support part 400.

That is, the heating part 200 is inserted into the inner space 110 of the ceramic part 100 in a state where the first bushing 120 is coupled to one side of the ceramic part 100. In this case, the heating part 200 may be inserted in a state where the heating element 210 and the transfer member 220 are assembled with each other, or sequentially inserted in a state where the heating element 210 and the transfer member 220 are separated.

As illustrated in FIG. 2, the transfer member 220 may include a first transfer body 221 and a second transfer body 222 that are disposed to face each other, and the above-described heating element 210 may be provided between the first transfer body 221 and the second transfer body 222.

That is, by disposing the first transfer body 221 to contact on one side surface of the heating element 210, and the second transfer body 222 to contact on the other side surface of the heating element 210, the heat generated by the heating element 210 may be transferred to the first transfer body 221 and the second transfer body 222, and the heat that has been transferred to the first transfer body 221 and the second transfer body 222 is transferred to the ceramic part 100. A first contact surface 221d and a second contact surface 222d for contacting the inner peripheral surface of the inner space 110 may be formed on the outer peripheral surface of the first transfer body 221 and the outer peripheral surface of the second transfer body 222, respectively.

As described above, if the heat generated through the heating element 210 is configured to be directly transferred to the ceramic part 100 in a conductive manner through the first transfer body 221 and the second transfer body 222, the heat transfer performance may be improved such that the thermotherapy effect is enhanced.

In this case, as illustrated in FIG. 2, the power supply part 300 may have a first electrode 311 which is disposed on one side of the ceramic part 100 and a second electrode 312 which is disposed on the other side of the ceramic part 100.

By configuring such that the current supplied through the power supply part 300 moves to the heating element 210 through the first electrode 311 and the current passing through the heating element 210 moves back to the power supply part 300 through the second electrode 312, it is possible to enable a smooth current supply.

The first electrode 311 and the second electrode 312 are fixed to a non-rotating or low-speed rotation state through the support part 400, and they are in electrical contact with the heating part 200 to supply a current to the heating part 200 that rotates together with the ceramic part 100. That is, relative rotation occurs between the first electrode 311 and the heating part 200 and between the second electrode 312 and the heating part 200, and in this way, curved surfaces protruding toward the heating part 200 may be formed on the first electrode 311 and the second electrode 312 such that the current stably moves even during the process of relative rotation.

That is, as the curved surfaces are formed on the first electrode 311 and the second electrode 312, the heating part 200, the first electrode 311 and the second electrode 312 are electrically connected to each other in a point-contact manner, and as the first electrode 311 and the second electrode 312 come into contact with the heating part 200 in a point contact manner, it is possible to prevent noise generation due to friction, and it is possible to prevent the abrasion of the first electrode 311 and the second electrode 312.

A rotating ball bearing and a bearing housing surrounding the same may be provided at the ends of the first electrode 311 and the second electrode 312, and the current which is supplied through the power supply part 300 moves to the heating part 200 while sequentially passing through the bearing housing and the ball bearing. In addition, as the ball bearing is provided, even when the heating part 200 rotates, the ball bearing rotates together, thereby enabling a stable current supply.

The electrical contact method between the heating part 200 and the first electrode 311 and the second electrode 312 is not necessarily limited to a point contact manner, and even if it is a line contact manner or a surface contact manner, it may be sufficiently applied if noise generation or abrasion due to friction can be prevented.

Furthermore, the curved surfaces of the first electrode 311 and the second electrode 312 may be elastically pressed to move toward the heating part 200. This prevents the electrical contact from breaking while being spaced apart from each other as unintentional abrasion occurs on the heating part 200 or the first electrode 311 and the second electrode 312 in the process of using the thermotherapy device for a long period of time.

In this case, as illustrated in FIG. 2, an insulating member 230 may be provided between the first transfer body 221 and the second transfer body 222.

The current supplied through the power supply part 300 sequentially moves through the first electrode 311, the first transfer body 221, the heating element 210, the second transfer body 222 and the second electrode 312, and if the first transfer body 221 and the second transfer body 222 are in direct contact with each other, a short circuit may occur and the current supply through the power supply part 300 may not be performed, and thus, direct electrical contact between the first transfer body 221 and the second transfer body 222 may be effectively prevented through the insulating member 230.

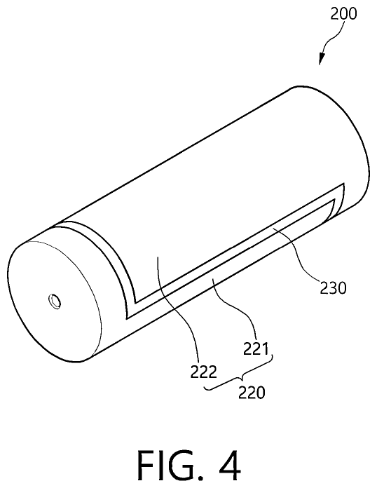

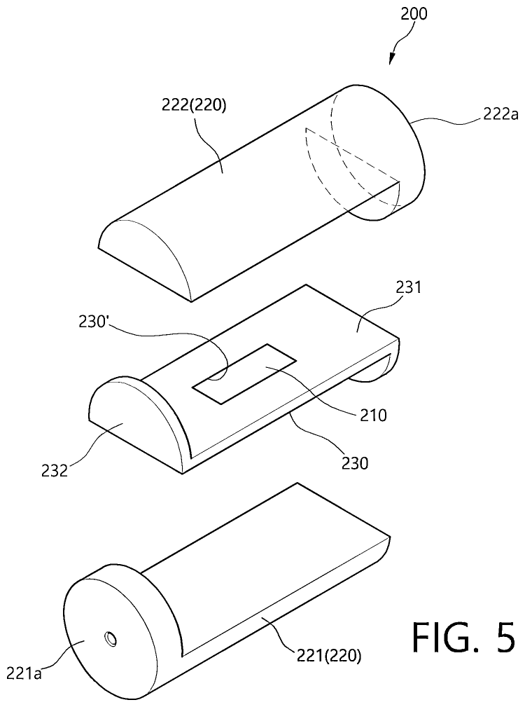

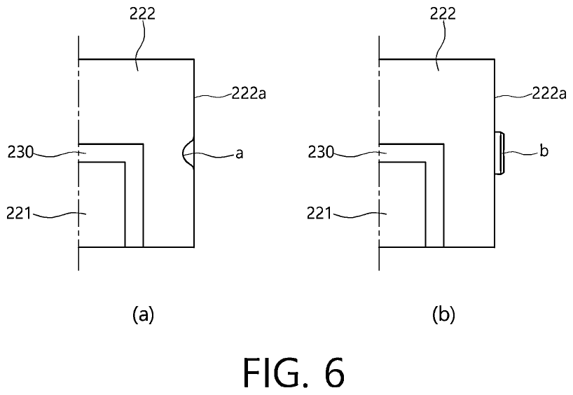

FIG. 4 is a perspective view illustrating a heating part according to an exemplary embodiment of the present invention, FIG. 5 is an exploded perspective view illustrating a heating part according to an exemplary embodiment of the present invention, and FIG. 6 is a cross-sectional view illustrating a conductive surface which is formed on a heating part according to an exemplary embodiment of the present invention.

As illustrated in FIG. 4, an insulating member 230 is provided between the first transfer body 221 and the second transfer body 222, and as illustrated in FIG. 5, an insertion groove 230′ into which the heating element 210 is inserted may be formed in the insulating member 230.

As such, when the heating element 210 is inserted and fixed into the insertion groove 230′, not only the heating element 210 can be disposed in the correct position, but also the heating element 210 is disposed in the insulating member 230 in a modular manner, and thus, it is possible to simplify the assembly process of the heating part 200.

The insertion groove 230′ is formed to pass through one side surface and the other side surface of the insulating member 230, and as illustrated in FIG. 2, while the heating element 210 is inserted into the insertion groove 230′, both side surfaces of the heating element 210 are exposed to contact the first transfer body 221 and the second transfer body 222. That is, in order for both side surfaces of the heating element 210 to come into contact with the first transfer body 221 and the second transfer body 222, based on the direction in which the heating element 210 is inserted into the insertion groove 230′, it is important to configure the heating element 210 to be disposed in the center of the insertion groove 230′ without being biased to either side.

To this end, the insertion groove 230′ may be provided with a separate stopper for fixing the position such that the heating element 210 can be disposed in the center of the insertion groove 230′.

In this way, when the heating element 210 is disposed on the insulating member 230 in a modular manner, direct electrical contact between the first transfer body 221 and the second transfer body 222 is prevented, but the current supplied while sequentially passing through the first electrode 311 and the first transfer body 221 moves to the heating member 210, and through this, heat is generated in the heating element 210, and then, the current moves to the power supply part 300 while sequentially passing through the second transfer body 222 and the second electrode 312.

As illustrated in FIG. 5, a first conductive surface 221a which is in electrical contact with the first electrode 311 may be formed on the first transfer body 221, and a second conductive surface 222a which is in electrical contact with the second electrode 312 may be formed on the second transfer body 222.

The current supplied from the power supply part 300 moves to the first transfer body 221 through the first conductive surface 221a conducting electricity with the first electrode 311 and then is supplied to the heating element 210, and the current that has passed through the heating element 210 moves to the second transfer body 222 and then moves back to the power supply part 300 through the second conductive surface 222a conducting electricity with the second electrode 312.

In this case, as described above, the heat generated by the heating element 210 is transferred to the ceramic part 100 through the first transfer body 221 and the second transfer body 222. That is, the first transfer body 221 and the second transfer body 222 provide a path for the current supplied through the power supply part 300 to move to the heating element 210, and at the same time, they transfer heat generated in the heating element 210 to the ceramic part 100, and therefore, the first transfer body 221 and the second transfer body 222 are preferably formed of a material in which current and heat can move at the same time. For example, when the first transfer body 221 and the second transfer body 222 are formed of an aluminum material, the number of free electrons inside the aluminum is large such that the movement of current and the transfer of heat may be made smoothly. In addition, the present invention is not necessarily limited to such an aluminum material, and as long as it is an alloy material of aluminum and magnesium, or a material which is capable of transferring current and heat at the same time, such as gold, silver, tungsten and copper, various materials may be used to form the first transfer body 221 and the second transfer body 222. In this case, if a coating such as graphene and the like is added to the first transfer body 221 and the second transfer body 222 through additional processing, the movement of current and the transfer of heat become smoother, and the efficiency may be further increased.

In this case, as illustrated in FIG. 5, the first conductive surface 221a is formed to extend to surround the second transfer body 222, and the second conductive surface 222a is formed to extend to surround the first transfer body 221.

That is, on one side of the heating part 200 on which the first electrode 311 is disposed, the first conductive surface 221a surrounds all of the second transfer body 222, and on the other side of the heating part 200 on which the second electrode 312 is disposed, the second conductive surface 222a surrounds all of the first transfer body 221, and thus, even when problems in which the heating part 200 is arbitrarily detached occur in the process of using the thermotherapy device, it is possible to stably prevent the first electrode 311 from conducting electricity with the second transfer body 222 or the second electrode 312 from conducting electricity with the first transfer body 221.

Moreover, as described above, the first conductive surface 221a is disposed to surround the second transfer body 222, and the second conductive surface 222a is disposed to surround the first transfer body 221, but it is necessary to prevent the first conductive surface 221a and the second transfer body 222 or the second conductive surface 222a and the first transfer body 221 from being electrically connected to each other.

To this end, the above-described insulating member 230 may be formed with a base surface 231 which is disposed between the first transfer body 221 and the second transfer body 222, and bent surfaces 232 which are disposed between the first conductive surface 221a and the second transfer body 222 and between the second conductive surface 222a and the first transfer body 221, respectively.

It is preferable that a conducting structure for stable conduction with the first electrode 311 and the second electrode 312 is formed on the first conductive surface 221a and the second conductive surface 222a.

That is, as illustrated in (a) of FIG. 6, a groove-shaped conductive groove (a) may be formed. Since the first electrode 311 and the second electrode 312 are respectively inserted into the conductive groove (a), it is possible to effectively prevent the heating part 200 from being separated.

Alternatively, as illustrated in (b) of FIG. 6, a protrusion-shaped conducting protrusion (b) may be formed. The first electrode 311 and the second electrode 312 may be formed with corresponding grooves into which the conductive protrusions (b) can be inserted, and since these conductive protrusions (b) are inserted into the corresponding grooves, it is possible to effectively prevent the heating part 200 from being separated.

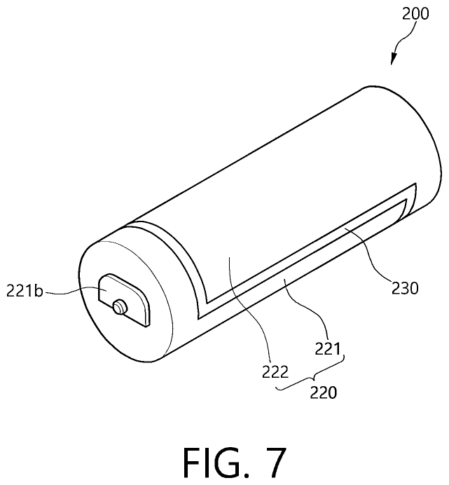

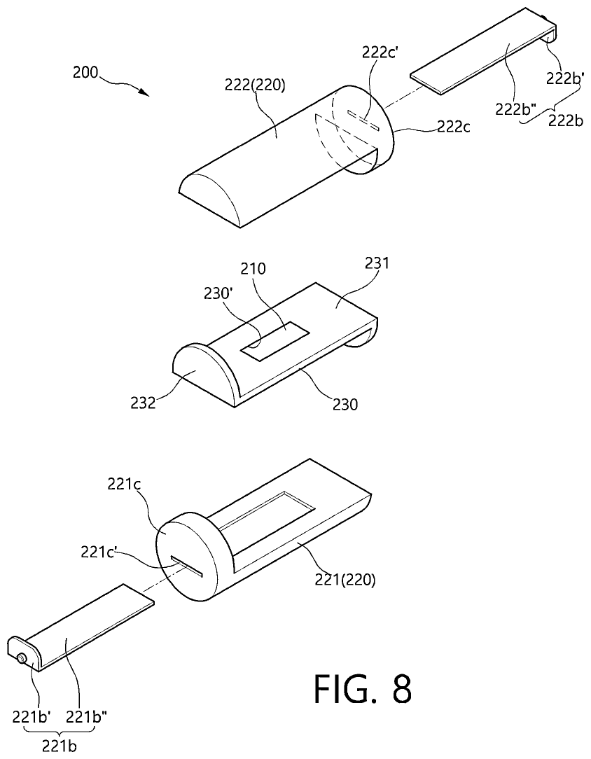

FIG. 7 is a perspective view illustrating a heating part according to another exemplary embodiment of the present invention, and FIG. 8 is an exploded perspective view illustrating a heating part according to another exemplary embodiment of the present invention.

As illustrated in FIG. 7, a separate first electrode plate 221b which is in electrical contact with the first electrode 311 may be formed on the first transfer body 221, and a second electrode plate 222b which is in electrical contact with the second electrode 312 may be formed on the second transfer body 222.

That is, the current supplied from the power supply part 300 is supplied to the heating element 210 through the first electrode plate 221b conducting electricity with the first electrode 311, and the current passing through the heating element 210 moves back to the power supply part 300 through the second electrode plate 222b conducting electricity with the second electrode 312.

As described above, when it is configured such that the first electrode plate 221b and the second electrode plate 222b supply current to the heating element 210 and the first transfer body 221 and the second transfer body 222 transfer heat generated at the heating element 210, it becomes easy to select the material of each electrode plate and the transfer body. This is because there is no need to consider the transfer of heat when selecting each electrode plate material, and there is no need to consider the movement of current when selecting each transfer body material.

In this case, as illustrated in FIG. 8, a first head 221b′ which is in contact with the first electrode 311 and a first body 221b″ which is in contact with one side surface of the heating member 210 may be formed on the first electrode plate 221b, and a second head 222b′ which is in contact with the second electrode 312 and a second body 222b″ which is in contact with the other side surface of the heating member 210 may be formed.

That is, the current supplied through the first head 221b′ conducting electricity with the first electrode 311 moves to the heating element 210 through the first body 221b″, and the current passing through the heating element 210 moves to the second head 222b′ conducting electricity with the second electrode 312 through the second body 222b″.

As described above, the first head 221b′ and the second head 222b′ are preferably provided with a conduction structure for stable electric conduction with the first electrode 311 and the second electrode 312.

That is, as illustrated in FIG. 8, a protrusion-shaped electrode protrusion may be formed, and corresponding grooves into which the electrode protrusion may be inserted may be formed on the first electrode 311 and the second electrode 312. In addition, since these electrode protrusions are inserted into the corresponding grooves, it is possible to effectively prevent the separation of the heating part 200.

Alternatively, by configuring such that conductive grooves in the form of a groove is formed and the first electrode 311 and the second electrode 312 are respectively inserted into the conductive grooves, it is possible to effectively prevent the heating part 200 from being separated.

In addition, as illustrated in FIG. 8, a first support surface 221c that extends to surround the second transfer body 222 and has a first through-hole 221c′ formed such that the first head 221b′ is exposed to the outside may be formed on the first transfer body 221, and a second support surface 222c that extends to surround the first transfer body 221 and has a second through-hole 222c′ formed such that the second head 222b′ is exposed to the outside may be formed on the second transfer body 222.

That is, on one side of the heating part 200 on which the first electrode 311 is disposed, the first support surface 221c covers all of the second transfer body 222, and on the other side of the heating part 200 on which the second electrode 312 is disposed, the second support surface 222c covers all of the first transfer body 221 such that it can be supported to limit the insertion depth of the first head 221b′ or the second head 222b′. In addition, even when problems in which the heating part 200 is arbitrarily separated occur in the process of using the thermotherapy device, it is possible to stably prevent the first electrode 311 from conducting electricity with the second transfer body 222 or the second electrode 312 from conducting electricity with the first transfer body 221.

In addition, as described above, in order for the current to be supplied through the first electrode plate 221b and the second electrode plate 222b in a state where the first support surface 221c and the second support surface 222c are formed, stable current supply is possible by forming a first through-hole 221c′ on the first support surface 221c to expose the first head 221b′ to the outside, and a second through-hole 222c′ on the second support surface 222c to expose the second head 222b′ to the outside.

In this case, an arrangement groove which is formed to extend from the first through-hole 221c′ may be formed on the first transfer body 221 to insert the first body 221b″, and an arrangement groove which is formed to extend from the second through-hole 222c′ may be formed on the second transfer body 222 to insert the second body 221b″, and through this, stable fixed arrangement of the first electrode plate 221b and the second electrode plate 222b is possible.

In addition, as described above, the first support surface 221c is disposed to surround the second transfer body 222, and the second support surface 222c is disposed to surround the first transfer body 221, but it is necessary to prevent the first support surface 221c and the second transfer body 222 or the second support surface 222c and the first transfer body 221 from being electrically energized with each other. To this end, in the above-described insulating member 230, a base surface 231 which is disposed between the first transfer body 221 and the second transfer body 222 may be formed, and bent surfaces 232 which are respectively disposed between the first support surface 221c and the second transfer body 222 and between the second support surface 222c and the first transfer body 221 may be formed. In this case, since electricity is conducted to the heating element 210 through the first electrode plate 221b and the second electrode plate 222b, the first transfer body 221 and the second transfer body 222 do not need to be made of materials capable of conducting electricity in which current flows, and it is also possible to form only with a material capable of transferring heat generated from the heating element 210 to the ceramic part 100. In this case, even if the first support surface 221c and the second transfer body 222 or the second support surface 222c and the first transfer body 221 come into contact with each other, they are not energized, and thus, it is also possible to remove the bent surface 232 and use the flat insulating member 230 with only the base surface 231 remaining.

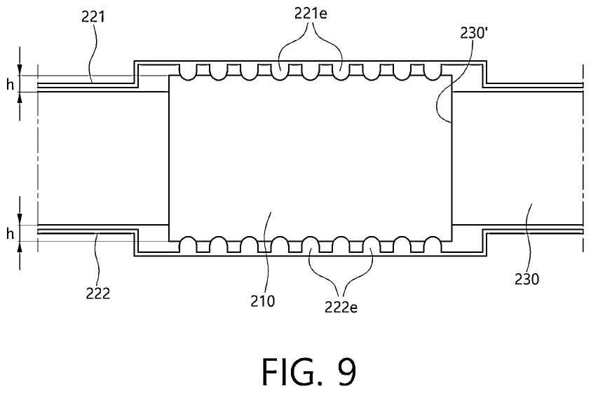

FIG. 9 is a cross-sectional view illustrating the coupling state of a heating element and a transfer member according to still another exemplary embodiment of the present invention.

As illustrated in FIG. 9, the heating element 210 may be formed to protrude by a predetermined height (h) such that both side surfaces thereof are exposed. That is, as both side surfaces of the heating element 210 are formed to protrude, electricity may be smoothly conducted with the first transfer body 221 and the second transfer body 222. Alternatively, even when current is supplied through the first electrode plate 221b and the second electrode plate 222b, both side surfaces of the heating element 210 are formed to protrude such that the first electrode plate 221b and the second electrode plate 222b can be smoothly energized. However, when the insulating member 230 is formed of an elastically deformable material, the heating element 210 and the insulating member 230 may be formed at the same height or the height of the heating element 210 may be formed to be lower than the height of the insulating member 230. In this case, when the first transfer body 221 and the second transfer body 222 press the insulating member 230 in the process of assembling the heating part 200, the insulating member 230 is elastically deformed, and accordingly, this is because the first transfer body 221 and the second transfer body 222 may be smoothly energized with the heating member 210. Even when the first electrode plate 221b and the second electrode plate 222b are provided, electricity may be smoothly conducted while the insulating member 230 is pressed.

In this case, as illustrated in FIG. 9, a first protrusion 221e may be formed on the first transfer body 221 to press one side surface of the heating element 210, and a second protrusion 222e may be formed on the second transfer body 221 to press the other side surface of the heating element 210.

In this way, when the first protrusion 221e and the second protrusion 222e are respectively formed, the heating element 210 may smoothly conduct electricity with the first transfer body 221 and the second transfer body 222. The first protrusion 221e and the second protrusion 222e may be formed on the heating element 210. Alternatively, when current is supplied through the first electrode plate 221b and the second electrode plate 222b, it is also possible to form the first protrusion 221e and the second protrusion 222e on the first electrode plate 221b and the second electrode plate 222b, respectively.

The first protrusion 221e and the second protrusion 222e are preferably configured to be elastically deformable. In a state where the heating part 200 is inserted and disposed in the inner space 110 of the ceramic part 100, if the outer peripheral surface of the heating part 200 is configured to be in surface contact with the inner peripheral surface of the inner space 110, heat may be smoothly transferred, and thus, the thermotherapy effect is improved, and heat loss is minimized such that the power consumption efficiency of the thermotherapy device is improved.

However, as the outer peripheral surface of the heating part 200 is in surface contact with the inner peripheral surface of the inner space 110, a frictional force is greatly applied to each other, and thus, it may not be easy to assemble the heating part 200, but when the first protrusion 221e and the second protrusion 222e are configured to be elastically deformable, the first protrusion 221e and the second protrusion 222e are elastically deformed in the process of assembling the heating part 200 such that not only can they be easily assembled, but also after the assembly of the heating part 200 is completed, the first protrusion 221e and the second protrusion 222e are elastically restored, and elastic force is applied such that the heating part 200 closely adheres to the ceramic part 100, thereby smoothly transferring heat.

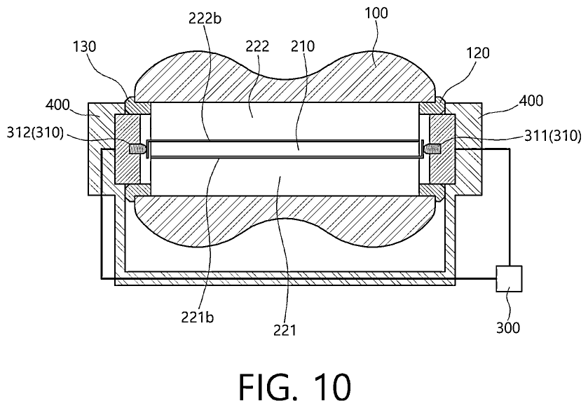

FIG. 10 is a cross-sectional view illustrating a state in which the ceramic part and the heating part are coupled according to still another exemplary embodiment of the present invention.

As illustrated in FIG. 10, it may be configured such that only the heating element 210 is disposed between the first electrode plate 221b and the second electrode plate 222b that are disposed to face each other, and a separate insulating member 230 is not used.

In this case, on the first electrode plate 221b and the second electrode plate 222b, heads that are respectively energized to the first electrode 311 and the second electrode 312 are formed to be bent, but the head formed on the first electrode plate 221b needs to be spaced apart from the second electrode plate 222b so as not to be energized with the second electrode plate 222b, and the head formed on the second electrode plate 221b needs to be spaced apart from the first electrode plate 222b so as not to be energized with the first electrode plate 221b.

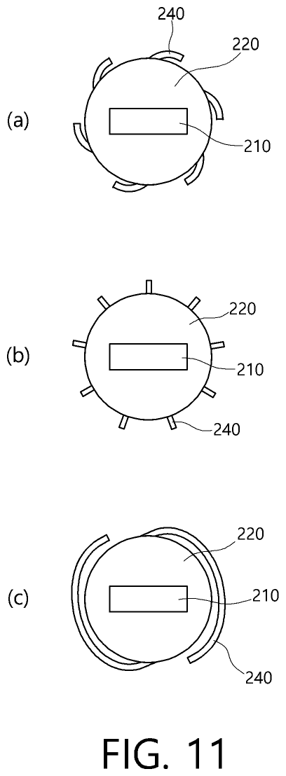

FIG. 11 is a side view illustrating a heating part according to still another exemplary embodiment of the present invention.

As illustrated in FIG. 111, the heating part 200 may include an elastically deformable member 240 for pressing the inner peripheral surface of the ceramic part 100. The elastically deformable member 240 is basically elastically deformed in the process of assembling the heating part 200 to the ceramic part 100, and it is elastically restored after the assembly to press the inner peripheral surface of the ceramic part 100. Therefore, since the heat generated by the heating part 200 can be directly transferred to the ceramic part 100 through the elastically deformable member 240 in a conductive manner, the thermotherapy effect is improved.

In this case, not only the heating part 200 but also the ceramic part 100 undergoes thermal expansion due to the heat generated through the heating part 200 in the process of using the thermotherapy device, and when the materials of the heating part 200 and the ceramic part 100 are different, the degree of thermal expansion is different. For example, when the ceramic part 100 is formed of a ceramic material and the heating part 200 is formed of an aluminum material, the degree of thermal expansion of the heating part 200 is greater than the degree of thermal expansion of the ceramic part 100, and as a result, in the process of using the thermotherapy device, phenomena in which the heating part 200 presses the inner peripheral surface of the ceramic part 100 may occur, and as a result, there may be problems in that the ceramic part 100 is damaged. Therefore, as described above, when the elastically deformable member 240 is provided in the heating part 200, the force pressing the inner peripheral surface of the ceramic part 100 is reduced as the elastically deformable member 240 is elastically deformed when the heating part 200 thermally expands, and thus, it is possible to effectively prevent the ceramic part 100 from being damaged.

At least one of these elastically deformable members 240 may be provided along the periphery of the heating part 200, and as illustrated in (a) of FIG. 11, it is preferable that the front end of the elastically deformable member 240 is disposed adjacent to the outer peripheral surface of the heating part 200 and configured to be spaced apart from the outer peripheral surface of the heating part 200 by a predetermined distance so as to be elastically deformable. When configured in this way, if thermal expansion occurs in the heating part 200, the elastically deformable member 240 is elastically deformed such that the separation distance between the front end of the elastically deformable member 240 and the outer peripheral surface of the heating part 200 decreases, and the force pressing the inner peripheral surface of the ceramic part 100 is reduced. Alternatively, as illustrated in (b) of FIG. 11, it is also possible to configure the elastically deformable member 240 to extend in the radial direction. When configured in this way, if thermal expansion occurs in the heating part 200, the elastically deformable member 240 is elastically deformed in a bending manner, and the force pressing the inner peripheral surface of the ceramic part 100 is reduced. Moreover, as illustrated in (c) of FIG. 11, it is also possible to configure the elastically deformable member 240 to partially cover the outer peripheral surface of the heating part 200. That is, for the basic operation in which the elastically deformable member 240 is elastically deformed during the thermal expansion of the heating part 200, (a) and (c) of FIG. 11 are similar, but the elastically deformable member 240 illustrated in (c) of FIG. 11 is formed to be longer than the elastically deformable member 240 illustrated in (a) of FIG. 11. When configured in this way, a contact area between the elastically deformable member 240 and the inner peripheral surface of the ceramic part 100 is increased such that the heat transfer effect can be improved.

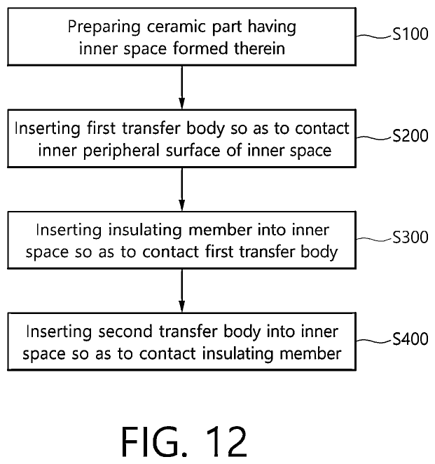

FIG. 12 is a flowchart illustrating the process of coupling a heating part to a ceramic part according to an exemplary embodiment of the present invention.

The ceramic part 100 having the inner space 110 formed therein is prepared (S100), and the heating part 200 is inserted into the inner space 110, and when the heating part 200 is inserted, the first transfer body 221, the insulating member 230 and the second transfer body 222 may all be inserted in an assembled state, or the first transfer body 221 is first inserted to contact the inner peripheral surface of the inner space 110 (S200), and after the insulating member 230 is inserted into the inner space 110 to contact the first transfer body 221 (S300), the second transfer body 222 is inserted into the inner space 110 so as to contact the insulating member 230 (S400).

In this way, when the heating part 200 is inserted in a separated state into the first transfer body 221, the insulating member 230 and the second transfer body 222, the magnitude of the frictional force acting between the inner peripheral surface of the inner space 110 and each component is reduced, and thus, it is possible to easily assemble the heating part 200.

Although an exemplary embodiment of the present invention has been described above, the spirit of the present invention is not limited to the exemplary embodiments presented herein, and a person skilled in the art who understands the spirit of the present invention may easily suggest other exemplary embodiments by modifying, changing, deleting or adding components within the scope of the same spirit, but it can be said that this will also fall within the spirit of the present invention.

1. A thermotherapy device, comprising:
a ceramic part having an inner space formed therein;
a heating part which is inserted in the inner space and has a heating element for generating heat so as to heat the ceramic part, and a transfer member for transferring the heat generated by the heating element to the ceramic part;
a power supply part for supplying a current to the heating part; and
a support part for supporting the ceramic part,
wherein the heating part can rotate relative to the power supply part such that the heating part rotates together with the ceramic part.

2. The thermotherapy device of claim 1, wherein the transfer member comprises a first transfer body and a second transfer body that are disposed to face each other, and
wherein the heating element is provided between the first transfer body and the second transfer body.

3. The thermotherapy device of claim 2, wherein the power supply part is provided with a first electrode which is disposed on one side of the ceramic part and a second electrode which is disposed on the other side of the ceramic part.

4. The thermotherapy device of claim 3, wherein an insulating member is provided between the first transfer body and the second transfer body.

5. The thermotherapy device of claim 4, wherein the insulating member is formed with an insertion groove into which the heating element is inserted and disposed, and
wherein both side surfaces of the heating element are exposed to contact with the first transfer body and the second transfer body.

6. The thermotherapy device of claim 4, wherein a first conductive surface which electrically contacts the first electrode is formed on the first transfer body, and
wherein a second conductive surface which electrically contacts the second electrode is formed on the second transfer body.

7. The thermotherapy device of claim 6, wherein the first conductive surface is formed to extend to surround the second transfer body,
wherein the second conductive surface is formed to extend to surround the first transfer body, and
wherein the insulating member is formed with a base surface which is disposed between the first transfer body and the second transfer body, and bent surfaces which are disposed between the first conductive surface and the second transfer body and between the second conductive surface and the first transfer body, respectively.

8. The thermotherapy device of claim 4, wherein a first electrode plate which electrically contacts the first electrode is formed on the first transfer body, and
wherein a second electrode plate which electrically contacts the second electrode is formed on the second transfer body.

9. The thermotherapy device of claim 8, wherein a first head which is in contact with the first electrode and a first body which is in contact with one side surface of the heating element are formed on the first electrode plate, and
wherein a second head which is in contact with the second electrode and a second body which is in contact with the other side surface of the heating element are formed on the second electrode plate.

10. The thermotherapy device of claim 9, wherein a first support surface which extends to surround the second transfer body and has a first through-hole formed such that the first head is exposed to the outside is formed on the first transfer body, and
wherein a second support surface which extends to surround the first transfer body and has a second through-hole formed such that the second head is exposed to the outside is formed on the second transfer body.

11. The thermotherapy device of claim 10, wherein the insulating member is formed with a base surface which is disposed between the first transfer body and the second transfer body, and bent surfaces which are disposed between the first support surface and the second transfer body and between the second support surface and the first transfer body, respectively.

12. The thermotherapy device of claim 5, wherein the heating element is formed to protrude by a certain height such that both side surfaces are exposed.

13. The thermotherapy device of claim 12, wherein a first protrusion is formed on the first transfer body to press one side surface of the heating element, and
wherein a second protrusion is formed on the second transfer body to press the other side surface of the heating element.

14. The thermotherapy device of claim 1, wherein the heating part is provided with an elastically deformable member that presses the inner peripheral surface of the ceramic part.
